(19) United States
(12) Patent Application Publication
(10) Pub. No.: US 2004/0241121 A1
(43) Pub. Date: Dec. 2, 2004

(54) Cosmetic composition comprising at least one polyester resulting from esterification of at least one triglyceride of hydroxylated carboxylic acid(s) and also comprising at least one pasty compound

(76) Inventors: Xavier Blin, Paris (FR); Vanina Filippi, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/797,051

(22) Filed: Mar. 11, 2004

Related U.S. Application Data
(60) Provisional application No. 60/456,228, filed on Mar. 21, 2003.
(60) Provisional application No. 60/456,230, filed on Mar. 21, 2003.

(30) Foreign Application Priority Data
Mar. 12, 2003 (FR) 03 03077
Mar. 12, 2003 (FR) 03 03079

(51) Int. Cl. A61K 7/06

(52) U.S. Cl. 424/70.11

(57) ABSTRACT

Disclosed herein is a cosmetic care or makeup composition comprising a) at least one polyester resulting from esterification of at least one triglyceride of at least one hydroxylated carboxylic acid with at least one aliphatic monocarboxylic acid and with at least one aliphatic dicarboxylic acid, and b) at least one pasty compound. This composition makes it possible to obtain a deposition on a keratin materials which can be lubricious, glossy, and comfortable, can have sharply defined outlines, does not migrate, and whose color intensity can be enhanced and/or whose color retention after challenge can be enhanced.

This application claims benefit of U.S. Provisional Application No. 60/456,228, filed Mar. 21, 2003, and U.S. Provisional Application No. 60/456,230, filed Mar. 21, 2003.

Disclosed herein is a cosmetic makeup or care composition for the skin, including the scalp, of both the human face and body, the lips or the epidermal derivatives of humans, such as hair, eyelashes, eyebrows and nails, which comprises, in a cosmetically acceptable medium, at least one specific polyester.

The composition disclosed herein comprises at least one polyester resulting from esterification of at least one triglyceride of at least one hydroxylated carboxylic acid with at least one aliphatic monocarboxylic acid and with at least one aliphatic dicarboxylic acid.

This composition can have notable cosmetic properties and can endow the makeup or care product with at least one property chosen from properties of, for example, gloss, lubricity on application, comfort, color retention over time and after challenge, gloss retention over time, non-migration, outline definition and color intensity.

The composition disclosed herein may, for example, constitute a makeup product for the body, lips or epidermal derivatives of humans which can have properties of, for example, non-therapeutic treatment and/or care. In one embodiment, the composition disclosed herein constitutes a lipstick or lipgloss, a blusher or eyeshadow, a tattooing product, a mascara, an eyeliner, a nail varnish, an artificial tanning product for the skin or a haircare or hair coloring product.

The present inventors have obtained, surprisingly, a composition comprising at least one polyester resulting from esterification of at least one triglyceride of at least one hydroxylated carboxylic acid with at least one aliphatic monocarboxylic acid and with at least one aliphatic dicarboxylic acid, which can be glossy, comfortable and does not migrate. Moreover, the composition's color intensity can be much better than that of the prior art compositions.

This composition can further exhibit effective dispersion of the pigments and/or fillers present in the composition; it does not exude when in stick form; it can have good properties of spreading and lubricity; and, moreover, it can endow the deposited film with sharply defined outlines and with properties of effective gloss retention and color retention over time (no color fading for at least three hours, uniform disappearance of the makeup). It can be, furthermore, stable, for example, for a number of months at ambient temperature (25° C. for more than a year) and can also be stable to heat (47° C. for 2 months) and to ultraviolet light without breakdown or odor over time.

Disclosed herein, therefore, is a composition comprising a) at least one polyester resulting from esterification of at least one triglyceride of at least one hydroxylated carboxylic acid with at least one aliphatic monocarboxylic acid and with at least one aliphatic dicarboxylic acid, and b) at least one pasty compound.

Polyester from at Least One Triglyceride of at Least One Hydroxylated Aliphatic Acid

The composition disclosed herein comprises at least one polyester resulting from esterification of at least one triglyceride of at least one hydroxylated carboxylic acid with at least one aliphatic monocarboxylic acid and with at least one aliphatic dicarboxylic acid, which is optionally unsaturated.

The at least one hydroxylated carboxylic acid disclosed herein is chosen from aliphatic hydroxycarboxylic acids. The at least one triglyceride of the at least one hydroxylated carboxylic acid disclosed herein is a glycerol substituted by three hydroxylated carboxylic acid residues, which can be identical or different. For example, a triglyceride obtained by reacting one equivalent of glycerol and three equivalents of a hydroxylated carboxylic acid can be called “triglyceride of hydroxylated carboxylic acid”. A triglyceride obtained by reacting one equivalent of glycerol with three equivalents of a mixture of at least two different hydroxylated carboxylic acids can be called “triglyceride of hydroxylated carboxylic acid(s).” Both types belong to the category “triglyceride of at least one hydroxylated carboxylic acid” as disclosed herein.

In one embodiment, the at least one polyester disclosed herein is liquid at ambient temperature (generally ranging from 20° C. to 25° C.) and atmospheric pressure (760 mm Hg).

In another embodiment, the at least one polyester disclosed herein has a viscosity at 25° C. of more than 500 cP (50 Pa.s), such as ranging from 900 to 10 000 cP (90 to 1 000 Pa.s) and further such as ranging from 950 to 5 000 cP (95 to 500 Pa.s), measured with a Brookfield RV or Brookfield “DV-II+” viscometer of type LV equipped with a No. 1 spindle rotating at a speed ranging from 0.5 rpm to 10 rpm. This viscosity measurement is taken when the measurement value has stabilized, generally after 10 minutes.

In another embodiment, the at least one polyester disclosed herein has a refractive index greater than or equal to 1.47, such as from 1.47 to 1.55, and further such as from 1.48 to 1.55 (wherein the refractive index is defined for the sodium D line). The refractive index is measured at 20° C. by reference to D Ray sodium (589 nm) according to, for example, ASTM Standards D 1218-92, D1500.

According to one embodiment, the at least one polyester is obtained by two esterification reactions of at least one triglyceride of at least one hydroxylated carboxylic acid: one esterification with at least one aliphatic monocarboxylic acid and one esterification with at least one aliphatic dicarboxylic acid.

In this embodiment, the at least one polyester is obtained by:

a) esterifying at least one of the hydroxyl functional groups of a triglyceride of at least one hydroxylated carboxylic acid with an aliphatic monocarboxylic acid and

b) esterifying the remaining hydroxyl functional groups of the triglyceride of at least one hydroxylated carboxylic acid, i.e., those not esterified with the aliphatic monocarboxylic acid, with an aliphatic dicarboxylic acid.

The esterification with an aliphatic monocarboxylic acid is thus, in one embodiment, conducted before the esterification with an aliphatic dicarboxylic acid.

The at least one hydroxylated carboxylic acid (i.e., the precursor of the triglyceride of at least one hydroxylated carboxylic acid) is, for example, chosen from hydroxylated aliphatic carboxylic acids comprising from 6 to 40 carbon atoms, such as from 10 to 34 carbon atoms and further such as 12 to 28 carbon atoms, and even further such as from 16 to 20 carbon atoms. In one embodiment, the at least one hydroxylated carboxylic acid comprises 18 carbon atoms.

The at least one hydroxylated carboxylic acid is, for example, chosen from saturated and unsaturated fatty acids.

For example, the at least one hydroxylated carboxylic acid may be chosen from:

i) saturated linear monohydroxylated aliphatic monocarboxylic acids of formulae (1) and (2):

(1)

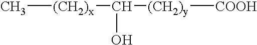

with 3≦x+y≦37

and (2) HO—CH₂—(CH₂)ₙ—COOH with 4≦x≦38;

ii) saturated branched monohydroxylated aliphatic monocarboxylic acids of formulae (3) and (3′):

(3)

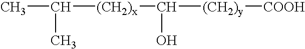

with 1≦x+y≦35

and (3′) 2-ethyl-3-hydroxycaprylic acid of formula

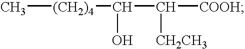

iii) unsaturated monohydroxylated aliphatic monocarboxylic acids of formulae (4), (5), and (6):

(4)

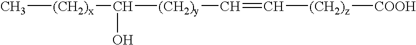

with 1≦x+y+z≦35

(5)

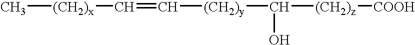

with 1≦x+y+z≦35

and (6) HOCH₂—(CH₂)ₙ—CH═CH—(CH₂)_{y}—COOH with 2≦x+y≦36;

iv) saturated polyhydroxylated aliphatic monocarboxylic acids of formula (7)

(7)

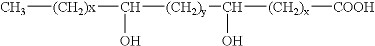

with 2≦x+y+z≦36

and the corresponding unsaturated polyhydroxylated aliphatic mbnocarboxylic acids;

v) saturated monohydroxylated aliphatic polyacids of formula (8)

(8)

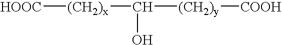

with 3≦x+y≦37

and the corresponding unsaturated monohydroxylated aliphatic polyacids; and

vi) saturated and unsaturated polyhydroxylated aliphatic polyacids.

In one embodiment, the at least one hydroxylated carboxylic acid is chosen from:

12-hydroxystearic acid, α-hydroxyoctadecanoic acid, hydroxy-14-eicosenoic acid;

leucinic acid, 2-ethyl-3-hydroxycaprylic acid;

ricinoleic acid;

3-hydroxy-4-hexanoic acid, oxynervonic acid;

16-hydroxy-6-hexadecenoic acid; and

9,10-dihydroxyoctadecanoic acid, 9,12-dihydroxyoctadecanoic acid, aleuritic acid, 9,10,12-trihydroxyoctadecanoic acid, hexahydroxyoctadecanoic acid and octahydroxyoctadecanoic acid.

The at least one hydroxylated carboxylic acid may, for example, be chosen from unsaturated fatty acids comprising from 16 to 20 carbon atoms, such as 18 carbon atoms.

The at least one triglyceride disclosed herein may be, for example, triglyceride of ricinoleic acid. This triglyceride is present in large amounts in the natural state in castor oil.

The at least one triglyceride of at least one hydroxylated carboxylic acid disclosed herein may, for example, be chosen from triglycerides of at least one hydroxylated carboxylic acid such that the at least one hydroxylated carboxylic acid comprises from 6 to 40 carbon atoms, such as from 10 to 34 carbon atoms, and further such as from 12 to 28 carbon atoms, and even further such as from 16 to 20 carbon atoms. In one embodiment, the at least one hydroxylated carboxylic acid comprises 18 carbon atoms.

The at least one aliphatic monocarboxylic acid disclosed herein may be chosen from saturated and unsaturated aliphatic fatty acids, such as isostearic acid.

The at least one aliphatic dicarboxylic acid disclosed herein comprises, for example, from 3 to 10 carbon atoms, such as from 3 to 6 carbon atoms, and further such as from 3 to 5 carbon atoms. According to one embodiment, the at least one aliphatic dicarboxylic acid is chosen from those of the formula HOOC—(CH₂)ₙ—COOH wherein n=1 to 4.

For example, the at least one aliphatic dicarboxylic acid may be succinic acid, corresponding to the above formula wherein n=2.

In one embodiment, the at least one polyester disclosed herein is of the formula (I)

T₂O-(OC-D-CO-O-T₁-O)ₓ-OC-D-CO-OT₂ (I)

wherein

The T₂-O- and -O-T₂ portions of the at least one polyester are derived from T₂-OH, which is a triglyceride of at least one hydroxylated carboxylic acid, wherein the triglyceride has been esterified with two molecules of an aliphatic monocarboxylic acid, and the triglyceride comprises a single free hydroxyl functional group;

The O-T₁-O- portion of the at least one polyester is derived from OH-T₁—OH, which is a triglyceride of at least one hydroxylated carboxylic acid, wherein the triglyceride has been esterfied with one molecule of an aliphatic monocarboxylic acid, and the triglyceride comprises two free hydroxyl functional groups;

The -OC-D-CO- portion of the at least one polyester is derived from HOOC-D-COOH, which is the at least one dicarboxylic acid; and

x ranges from 1 to 50, such as from 1 to 10, and further such as from 2 to 6.

x can be chosen, for example, from 3, 4, 5, 6, 7, 8, 9, and 10.

The at least one polyester disclosed herein can be chosen, for example, from the polyesters described in U.S. Pat. No. 6,342,527, the content of which is incorporated by reference into the present application. The at least one polyester disclosed herein can, for example, be of the formula (I) above wherein

T₂O- is

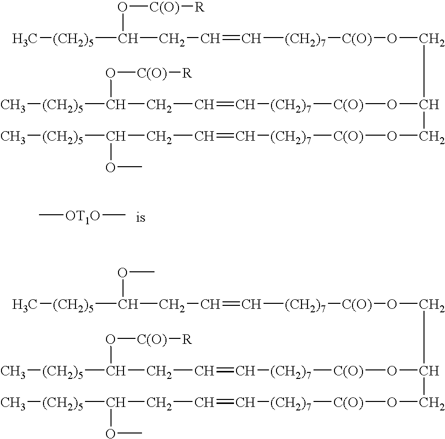

In the above two formulae, R is chosen from alkyl and alkylene groups comprising from 5 to 33 carbon atoms.

In one embodiment, R is chosen from alkyl groups comprising from 7 to 17 carbon atoms and alkylene groups comprising from 11 to 21 carbon atoms.

The at least one polyester disclosed herein may be present in an amount ranging from 0.1% to 99.9% by weight, such as from 1% to 99% by weight, further such as from 1% to 80% by weight, and further such as from 10% to 40% by weight, further such as from 15% to 25% by weight, and even further such as from 20% to 25% by weight of the total weight of the composition. In general, the at least one polyester can be present in an amount sufficient to endow the composition with at least one property chosen from properties of gloss, stability, color retention over time, gloss retention, comfort, non-migration and outline definition following application of the composition.

Pasty Compound

As used herein, the term “pasty compound” means a lipophilic fatty compound, which exhibits a reversible solid/liquid state change and, in the solid state, has an anisotropic crystalline organizational structure, and which, at a temperature of 23° C., comprises a liquid fraction and a solid fraction.

The at least one pasty compound disclosed herein may be a compound having a hardness at 20° C. ranging from 0.001 to 0.5 MPa, such as from 0.002 to 0.4 MPa.

The hardness is measured in accordance with a method involving penetration of a probe into a sample of compound, and, for example, with the aid of a texture analyser (for example, the TA-XT2i from Rheo) equipped with a stainless steel cylinder 2 mm in diameter. The hardness measurement is made at 20° C. in the center of 5 samples. The cylinder is introduced into each sample at a preliminary speed of 1 mm/s and then at a measurement speed of 0.1 mm/s, wherein the depth of penetration is 0.3 mm. The value taken for the hardness is that of the maximum peak.

Moreover, at a temperature of 23° C., the at least one pasty compound is in the form of a liquid fraction and a solid fraction. In other words, the incipient melting temperature of the at least one pasty compound is less than 23° C. The liquid fraction of the at least one pasty compound, measured at 23° C., ranges from 9% to 97% by weight of the total weight of the pasty compound. This liquid fraction at 23° C. may also range, for example, from 15% to 85% by weight, such as from 40% to 85% by weight of the total weight of the pasty compound.

The liquid fraction by weight of the at least one pasty compound at 23° C. is equal to the ratio of the enthalpy of fusion which is consumed at 23° C. to the enthalpy of fusion of the pasty compound.

The enthalpy of fusion of the pasty compound is the enthalpy consumed by the compound in passing from the solid state to the liquid state. The pasty compound is said to be in the solid state when the entirety of its mass is in crystalline solid form. The pasty compound is said to be in the liquid state when the entirety of its mass is in liquid form.

The enthalpy of fusion of the pasty compound is equal to the area under the curve of the thermogram obtained by means of a differential scanning calorimeter (DSC) such as the calorimeter sold under the name MDSC 2920 by the company TA instrument, with a temperature rise of 5° or 10° C. per minute, in accordance with standard ISO 11357-3:1999. The enthalpy of fusion of the pasty compound is the amount of energy required to cause the compound to pass from the solid state to the liquid state, and is expressed in J/g.

The enthalpy of fusion consumed at 23° C. is the amount of energy absorbed by the sample in passing from the solid state to the state which the sample has at 23° C., consisting of a liquid fraction and a solid fraction.

The liquid fraction of the pasty compound, measured at 32° C., ranges, for example, from 30% to 100% by weight, such as from 80% to 100% by weight, and further such as from 90% to 100% by weight of the total weight of the pasty compound. When the liquid fraction of the pasty compound, measured at 32° C., is 100%, the temperature of the end of the melting range of the pasty compound is less than or equal to 32° C.

The liquid fraction of the pasty compound, measured at 32° C., is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The at least one pasty compound disclosed herein may be, for example, chosen from synthetic compounds and compounds of vegetable origin. The at least one pasty compound may be obtained by synthesis from starting materials of vegetable origin.

In one embodiment, the at least one pasty compound may be chosen from:

lanolin and its derivatives;

polymeric and non-polymeric silicone compounds;

polymeric and non-polymeric fluoro compounds;

vinyl polymers chosen, for example, from

olefin homopolymers,

olefin copolymers,

hydrogenated diene homopolymers and copolymers,

linear and branched oligomers, homopolymers and copolymers of alkyl (meth)acrylates, such as those comprising at least one alkyl group chosen from C₈-C₃₀ alkyl groups,

oligomers, homopolymers and copolymers of vinyl esters comprising at least one alkyl group chosen from C₈-C₃₀ alkyl groups,

oligomers, homopolymers and copolymers of vinyl ethers comprising at least one alkyl group chosen from C₈-C₃₀ alkyl groups;

fat-soluble polyethers resulting from polyetherification of at least one diol chosen from C₂-C₁₀₀ diols, such as C₂-C₅₀ diols, and esters.

The at least one pasty compound may, for example, be polymeric, e.g., such as a hydrocarbon compound.

In one embodiment, the at least one pasty compound is chosen from fluorosilicone pasty compounds, such as the polymethyltrifluoropropylmethylalkyldimethyl-siloxane sold under the name X22-1088 by SHIN ETSU.

When the at least one pasty compound is chosen from silicone and fluoropolymers, the composition disclosed herein may, for example, include at least one compatibilizer chosen, for example, short-chain esters, such as isodecyl neopentanoate.

Among the fat-soluble polyethers, examples include copolymers of at least one oxide chosen from ethylene oxide and propylene oxide with at least one alkylene oxide chosen from C₆-C₃₀ long-chain alkylene oxides, such as those wherein the weight ratio of the at least one oxide chosen from ethylene oxide and propylene oxide to the at least one alkylene oxide in the copolymer ranges from 5:95 to 70:30. Within this class, mention may be made, for example, of copolymers wherein the long-chain alkylene oxides are arranged in blocks having an average molecular weight ranging from 1,000 to 10,000; for example, a block copolymer of polyoxyethylene/polydodecyl glycol, such as the ethers of dodecanediol (22 mol) and of polyethylene glycol (45 EO) sold under the brand name ELFACOS ST9 by Akzo Nobel.

Among the esters, examples include:

esters of an oligomeric glycerol, such as diglycerol esters, for example, condensates of adipic acid and glycerol, wherein at least one hydroxyl group of the glycerol has reacted with a mixture of fatty acids such as stearic acid, capric acid, stearic acid and isostearic acid and 12-hydroxystearic acid, for example, the ester sold under the brand name SOFTISAN 649 by the company Sasol;

arachidyl propionate sold under the brand name WAXENOL 801 by Alzo;

phytosterol esters;

fatty acid triglycerides and their derivatives;

pentaerythritol esters;

non-crosslinked polyesters resulting from polycondensation of at least one acid chosen from linear and branched C₄-C₅₀ dicarboxylic and polycarboxylic acids and at least one entity chosen from C₂-C₅₀ diols and polyols; and.

aliphatic ester esters resulting from esterification of at least one aliphatic hydroxycarboxylic ester with at least one aliphatic carboxylic acid.

The at least one aliphatic carboxylic acid comprises from 4 to 30 such as from 8 to 30 carbon atoms. It may, for example, be chosen from hexanoic acid, heptanoic acid, octanoic acid, 2-ethyl hexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid and mixtures thereof.

In one embodiment, the at least one aliphatic carboxylic acid is branched.

The aliphatic hydroxycarboxylic ester is, for example, that of at least one hydroxylated aliphatic carboxylic acid comprising from 2 to 40 carbon atoms, such as from 10 to 34 carbon atoms and further such as from 12 to 28 carbon atoms, and comprising from 1 to 20 hydroxyl groups, such as from 1 to 10 hydroxyl groups and further such as from 1 to 6 hydroxyl groups. The aliphatic hydroxycarboxylic ester may be chosen, for example, from:

a) partial and complete esters of saturated linear monohydroxylated aliphatic monocarboxylic acids;

b) partial and complete esters of unsaturated monohydroxylated aliphatic monocarboxylic acids;

c) partial and complete esters of saturated monohydroxylated aliphatic polycarboxylic acids;

d) partial and complete esters of saturated polyhydroxylated aliphatic polycarboxylic acids; and

e) partial and complete esters of C₂ to C₁₆ aliphatic polyols having undergone reaction with at least one acid chosen from mono- and polyhydroxylated aliphatic monocarboxylic and polycarboxylic acids;

and mixtures thereof.

The aliphatic ester esters may, for example, be chosen from:

the ester resulting from the esterification reaction of hydrogenated castor oil with isostearic acid in proportions of 1 to 1 (1/1) or hydrogenated castor oil monoisostearate,

the ester resulting from the esterification reaction of hydrogenated castor oil with isostearic acid in proportions of 1 to 2 (1/2) or hydrogenated castor oil diisostearate,

the ester resulting from the esterification reaction of hydrogenated castor oil with isostearic acid in proportions of 1 to 3 (1/3) or hydrogenated castor oil triisostearate,

and mixtures thereof.

Among the pasty compounds of vegetable origin, examples include a mixture of soya sterols and of ethoxylated (5EO) and propoxylated (5 PO) pentaerythritol, which is sold as LANOLIDE by the company VEVY.

The at least one pasty compound is present in an amount ranging from 1% to 99% by weight, such as from 1% to 60% by weight, and further such as from 2% to 30% by weight, and even further such as from 5% to 15% by weight of the total weight of the composition.

In one embodiment, the composition disclosed herein is free of lanolin or one of its derivatives.

Forms of the Composition

The composition disclosed herein may be in the form of a solid composition, compacted or cast, as a stick or in a dish, or in paste or liquid form. In one embodiment, the composition disclosed herein is in a solid form, such as in a hard form (a form which does not flow under its own weight), for example, a cast or compacted form, such as a stick or dish.

The composition disclosed herein may be present in the form of a paste, solid or cream. It may be an oil-in-water or water-in-oil emulsion, an anhydrous gel which is solid or flexible, or may be in the form of a loose or compacted powder, and even in a two-phase form. In one embodiment, the composition disclosed herein is in the form comprising an oily, such as anhydrous, continuous phase; in this case, the composition may comprise an aqueous phase in a proportion of less than 10% by weight such as less than 5% by weight of the total weight of the composition.

Dyes

In one embodiment, the composition disclosed herein may further comprise at least one colorant, which may be chosen from dyes which are soluble or dispersible in the composition, pigments, nacres and mixtures thereof. The dyes are, for example, fat-soluble dyes, although water-soluble dyes can also be used. The at least one colorant may be present in an amount ranging from 0.001% to 98% by weight, such as from 0.5% to 85% by weight and further such as from 1% to 60% by weight of the total weight of the composition.

For a composition in paste or cast form, such as lipsticks or body makeup products, the at least one colorant is present in an amount ranging from 0.5% to 50% by weight, such as from 2% to 40% by weight and further such as from 5% to 30% by weight, relative to the total weight of the composition.

The fat-soluble dyes are chosen, for example, from Sudan Red, D & C Red 17, D & C Green 6, β-carotene, soya oil, Sudan Brown, D &. C Yellow 11, D & C Violet 2, D & C Orange 5, quinoline yellow and annatto. They may be present in an amount ranging from 0 to 20% by weight, such as from 0.1% to 6% by weight of the total weight of the composition. Water-soluble dyes are chosen, for example, from beet juice and methylene blue, and may be present in an amount ranging from 0.1 to 6% by weight of the total weight of the composition (if they are present).

In one embodiment, the composition disclosed herein comprises a particulate phase which, for example, is colored and may be present in an amount ranging from 0.001% to 50% by weight, such as from 0.01% to 40% by weight, and further such as from 0.05% to 30% by weight of the total weight of the composition. The particulate phase may comprise at least one ingredient chosen from pigments, nacres, and fillers, which are commonly used in cosmetic compositions.

As used herein, the term “pigments” means white or colored, organic or inorganic particles which are insoluble in the liquid fatty phase and are intended for coloring and/or opacifying the composition. The term “fillers” means colorless or white, mineral or synthetic particles which may be lamellar or non-lamellar. The term “nacres” means irridescent particles, which are produced, for example, by certain molluscs in their shell, or are synthesized. These fillers and nacres can serve, for example, to modify the texture of the composition.

The pigments may be present in an amount ranging from 0.05% to 30% by weight (if they are present), such as from 2% to 20% by weight of the total weight of the composition. As mineral pigments which can be used herein, mention may be made, for example, of titanium oxide, zirconium oxide and cerium oxide and also of zinc oxide, iron oxide, chromium oxide and ferric blue. Among the organic pigments which can be used herein, mention may be made, for example, of carbon black and lakes of barium, strontium, calcium (D & C Red No. 7) and aluminium.

The nacres may be present in an amount ranging from 0.001% to 20% by weight (if they are present), such as from 1% to 15% by weight of the total weight of the composition. Among the nacres which can be used herein, mention may be made, for example, of mica covered with titanium dioxide, iron oxide, natural pigment or bismuth oxychloride, such as colored titanium mica, goniochromatic pigments and, for example, multi-layer interference pigments.

The fillers may be present in an amount ranging from 0.001% to 35% by weight (if they are present), such as from 0.5% to 15% by weight of the total weight of the composition. Mention may be made, for example, of talc, mica, kaolin, Nylon® powder (ORGASOL in particular) and polyethylene powder, polytetrafluoroethylene (Teflon®) powders, starch, boron nitride, copolymer microspheres such as Expancel® (Nobel Industries), Polytrap® (Dow Corning), Polypore® L 200 (Chemdal Corporation) and silicone resin microbeads (such as Tospearl from Toshiba), and silica.

The composition disclosed herein may further comprise at least one additional non-aqueous compound chosen from oils and waxes.

Waxes

In one embodiment, the composition disclosed herein may comprise at least one wax.

As disclosed herein the term “wax” means a lipophilic fatty compound which is solid at ambient temperature (25° C.), exhibits a reversible solid/liquid state change, has a melting temperature greater than 30° C. and possibly up to 200° C., has a hardness greater than 0.5 MPa, and exhibits anisotropic crystalline organization in the solid state. By taking the wax to its melting temperature, it is possible to make it miscible with the oils and to form a microscopically homogeneous mixture, but by taking the temperature of the mixture to ambient temperature, the wax is recrystallized in the oils of the mixture.

The hardness can be measured according to the method known as the “cheesewire” method, which involves cutting a lipstick stick measuring 12.7 mm and measuring the hardness at 20° C. by means of a DFGHS 2 dynamometer from the company Indelco-Chatillon, travelling at a speed of 100 mm/minute. It is expressed as the shear force (expressed in grams) required to cut a stick under these conditions. In accordance with this method, the hardness of a stick composition disclosed herein ranges, for example, from 50 to 300 g, such as from 100 to 250 g and further such as from 150 to 230 g.

The waxes which can be used herein are compounds which are solid at room temperature and are intended to provide the composition with structure, such as in the form of a stick; they may be chosen from hydrocarbon waxes, fluoro waxes and silicone waxes and may be chosen from those of vegetable mineral, animal and synthetic in origin. For example, they have a melting temperature of greater than 40° C. and such as greater than 45° C.

As the wax which can be used herein, mention may be made of those waxes which are generally used in the cosmetics field: they are, for example, of natural origin, such as beeswax, carnauba wax, candelilla wax, ouricuri wax, Japan wax, cork fibre wax, sugarcane wax, rice wax, montan wax, paraffin, lignite wax, microcrystalline wax, ceresin, ozokerite, and hydrogenated oils such as jojoba oil; synthetic waxes, such as polyethylene waxes obtained from the polymerization or copolymerization of ethylene with a weight-average molecular mass ranging from 400 to 800 and mixtures thereof, Fischer-Tropsch waxes, and esters of fatty acids, such as octacosanyl stearate, glycerides which are solid at 40° C. or at 45° C., silicone waxes such as alkyl- and alkoxydimethicones comprising at least one chain chosen from alkyl and alkoxy chains of 10 to 45 carbon atoms, poly(di)methylsiloxane esters which are solid at 40° C. and whose ester chain comprises at least 10 carbon atoms; and mixtures thereof.

The nature and amount of the waxes are a function of the desired mechanical properties and textures. For example, the wax may be present in an amount ranging from 0.01% to 50% by weight, such as from 2% to 40% by weight and further such as from 5% to 30% by weight of the total weight of the composition.

Oils

An “oil,” as disclosed herein, is a fatty substance which is liquid at ambient temperature and atmospheric pressure.

The oils may be chosen from hydrocarbon oils, silicone oils and fluoro oils. These oils may be animal, vegetable, mineral or synthetic in origin.

A “hydrocarbon oil,” as disclosed herein is an oil which comprises primarily atoms of carbon and hydrogen and optionally at least one functional group chosen from hydroxyl, ester, ether and carboxyl functional groups. As examples of oils which can be used herein, mention may be made of the following:

hydrocarbon oils of animal origin such as perhydrosqualene;

vegetable hydrocarbon oils such as the liquid triglycerides of fatty acids of 4 to 24 carbon atoms, such as the triglycerides of heptanoic or octanoic acids, and sunflower oil, maize oil, soya oil, pumpkin oil, grapeseed oil, sesame oil, hazelnut oil, apricot oil, macadamia oil, castor oil, avocado oil, the triglycerides of caprylic/capric acids such as those sold by Stearineries Dubois and those sold under the names Miglyol 810, 812 and 818 by Dynamit Nobel, jojoba oil and shea butter;

linear or branched hydrocarbons of mineral or synthetic origin, such as liquid paraffins and their derivatives, vaseline, polydecenes and hydrogenated polyisobutene such as Parleam;

synthetic esters and ethers, such as those of fatty acids, for example, oils of formula R₁COOR₂ wherein R₁ is the residue of a higher fatty acid comprising from 1 to 40 carbon atoms and R₂ is a hydrocarbon chain comprising from 1 to 40 carbon atoms, with R₁+R₂≧10, such as Purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyidodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

fatty alcohols comprising from 12 to 26 carbon atoms, such as octyldodecanol, 2-butyloctanol, 2-hexyldecanol, 2-undecylpentadecanol and oleyl alcohol;

fluoro oils, optionally with partial hydrocarbon and/or silicone modification;

silicone oils such as volatile or non-volatile, linear or cyclic polydimethylsiloxanes (PDMS); polydimethylsiloxanes comprising at least one group chosen from alkyl, alkoxy and phenyl groups, pendantly or at the end of the silicone chain, wherein the at least one group comprises from 2 to 24 carbon atoms; phenyl silicones such as phenyltrimethicones, phenyldimethicones, phenyltrimethylsiloxydiphenylsiloxanes, diphenyldimethicones, diphenylmethyldiphenyltrisiloxanes and 2-phenylethyl trimethylsiloxysilicates; and

mixtures thereof.

The additional oils may be present in an amount ranging from 0 to 90% by weight, such as from 0.05% to 60% by weight, and further such as from 10% to 55% by weight of the total weight of the composition.

Additives

The composition disclosed herein may further comprise at least one additive chosen from additives commonly used in the field, such as water, antioxidants, preservatives, neutralizers, lipophilic gelling agents or liquid fatty substances, aqueous-phase gelling agents, dispersants and cosmetic or dermatological actives. These additives, with the exception of water, may be present in an amount ranging from 0 to 20% by weight, such as from 0 to 10% by weight of the total weight of the composition. Water may be present in an amount ranging from 0 to 70% by weight, such as from 1% to 50% by weight, and further such as from 1 to 10% by weight of the total weight of the composition,

The composition disclosed herein may be in the form of a colored or uncolored composition, in the form of a sun protection or makeup remover composition or in the form of a hygiene composition. The composition may include, for example, at least one cosmetic active material. It may therefore be used as a care base or treatment base for the skin, such as the hands or face, or for the lips (lip balms, which can protect the lips from cold, sun, and/or wind) or as a deodorant. The at least one cosmetic active material disclosed herein may be chosen, for example, from vitamins A, E, C and B₃, provitamins such as D-panthenol, active soothing agents such as α-bisabolol, aloe vera, allantoin, plant extracts and essential oils, protective and restructuring agents such as ceramides, active freshness agents such as menthol and its derivatives, emollients (cocoa butter, dimethicone), moisturizers (arginine PCA), active anti-wrinkle substances, and essential fatty acids.

The composition disclosed herein may also be in the form of a skin makeup product, for example, a face makeup product such as a foundation, a blusher, a rouge and an eyeshadow, a body makeup product such as a semi-permanent tattooing product, and a lip makeup product such as a lipstick and a lipgloss, possibly having treatment or care properties, a product for making up the epidermal derivatives, such as a nail varnish, a mascara and an eyeliner, and a haircare product and a hair coloring product.

The composition disclosed herein should be cosmetically acceptable, i.e., it should not be toxic and should be capable of application to the skin, epidermal derivatives or lips of human beings.

The composition disclosed herein may be manufactured by known methods which are generally employed in the cosmetic or dermatological field.

Further disclosed herein is a method for providing a deposit on a keratin material, by applying to the keratin material a cosmetic composition comprising (i) of at least one polyester resulting from esterification of at least one triglyceride of at least one hydroxylated carboxylic acid with at least one aliphatic monocarboxylic acid and with at least one aliphatic dicarboxylic acid, and (ii) of at least one pasty compound having, for example, a hardness at 25° C. ranging from 0.001 to 0.5 MPa, such as from 0.002 to 0.4 MPa, whose liquid fraction at 23° C. ranges, for example, from 9% to 97% by weight, such as from 15% to 85%, by weight, and further such as from 40% to 85% by weight of the total weight of the pasty compound, wherein the deposit may be in the form of a film, and wherein the deposit has at least one property chosen from properties of gloss, comfort, color retention over time, gloss retention over time when colored, non-migration, effective spreading and lubricity on application, limiting the exudation and enhancing the outline definition of the film, and enhancing the color intensity of the film when colored.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result. The amounts are given as percentages by weight of the total weight of the composition.

### EXAMPLE 1

### Lipstick

| | |
|---|---|
| Castor oil ester of succinic acid and isostearic acid (sold as | 22 |
| Zenigloss by Zenitech) | |
| Ethers of dodecanediol (22 mol) and of polyethylene glycol | 11 |
| (45 EO) (sold as Elfacos ST9 by Akzo Nobel) | |
| Triglyceride of 2-decyltetradecanoic acid | 20 |
| Hydrogenated polyisobutene | 10 |
| Diisostearyl malate | 11 |
| Polybutylene | 2.5 |
| Octacosanyl stearate | 5 |
| Mixture of triglycerides of lauric, myristic, palmitic and | 2 |
| stearic acids (50/20/10/10) | |
| Polyethylene wax | 5 |
| Hectorite (modified with distearyl- dimethylammonium | 3 |
| chloride) | |
| Pigments | qs |
| Preservative | qs |
| Fragrance | qs |

The oily phase was produced by mixing the preservative, all the oils and the paste (ethers of dodecanediol (22 mol) and of polyethylene glycol (45 EO)).

Then the hectorite was ground in the oily phase on a triple-roll mill.

The pigments were subsequently ground in the mixture of the hectorite and the oily phase.

The resulting mixture was added to a pan together with the waxes and the mixture was heated at 105° C. for two hours, with homogenization using a Rayneri apparatus.

Finally the fragrance was added and the mixture was homogenized for 5 minutes and then poured into a mold at 42° C. which was cooled at −20° C. for 30 minutes. The sticks were then demolded.

The above formula exhibited good gloss retention at 1 hour and did not migrate at 1 hour. It additionally possessed good properties in terms of application (lubricity), comfort, gloss (on application and over time) and color retention after challenge.

### EXAMPLE 2

### Lipstick

| | |
|---|---|
| Castor oil ester of succinic acid and isostearic acid (sold as | 24 |
| Zenigloss by Zenitech) | |
| Poly(bisdiglyceryl 2-acyladipate | 12 |
| (sold as Softisan 649 by Sasol) | |
| Sesame oil | 36.1 |
| Polyglycerolated beeswax (Cera Bellina sold by Koster | 4.2 |
| Keunen) | |
| Microcrystalline wax | 10.5 |
| Hectorite (modified with distearyldimethylammonium | 0.6 |
| chloride) | |
| Pigments | qs |
| Preservative | qs |
| Fragrance | qs |

The above formula possessed good properties in terms of application (lubricity), comfort and gloss (on application and over time).

What is claimed is:

1. A cosmetic composition comprising
i) at least one polyester resulting from esterification of at least one triglyceride of at least one hydroxylated carboxylic acid
a) with at least one aliphatic mbnocarboxylic acid and
b) with at least one aliphatic dicarboxylic acid, and
ii) at least one pasty compound.

2. The composition according to claim 1, wherein said at least one polyester is obtained by
a) esterifying at least one hydroxyl functional group of said at least one triglyceride with said at least one aliphatic monocarboxylic acid and
b) esterifying the remaining hydroxyl functional groups of said at least one triglyceride with said at least one aliphatic dicarboxylic acid.

3. The composition according to claim 1, wherein the at least one triglyceride of at least one hydroxylated carboxylic acid is chosen from triglycerides of at least one hydroxylated carboxylic acid wherein said at least one hydroxylated carboxylic acid comprises from 6 to 40 carbon atoms.

4. The composition according to claim 3, wherein the at least one hydroxylated carboxylic acid comprises from 10 to 34 carbon atoms.

5. The composition according to claim 4, wherein the at least one hydroxylated carboxylic acid comprises from 12 to 28 carbon atoms.

6. The composition according to claim 5, wherein the at least one hydroxylated carboxylic acid comprises from 16 to 20 carbon atoms.

7. The composition according to claim 6, wherein the at least one hydroxylated carboxylic acid comprises 18 carbon atoms.

8. The composition according to claim 1, wherein the at least one triglyceride of at least one hydroxylated carboxylic acid is chosen from triglycerides of at least one hydroxylated carboxylic acid chosen from
i) saturated linear monohydroxylated aliphatic monocarboxylic acids of formulae (1) and (2):
(1) 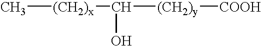
with 3≦x+y≦37
and (2) HO—CH₂—(CH₂)ₙ—COOH with 4≦x≦38;
ii) saturated branched monohydroxylated aliphatic monocarboxylic acids of formulae (3) and (3′):
(3) 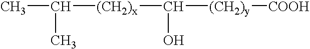
with 1≦x+y≦35
and (3′) 2-ethyl-3-hydroxycaprylic acid of formula 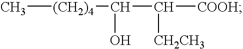
iii) unsaturated monohydroxylated aliphatic monocarboxylic acids of formulae (4), (5), and (6):
(4) 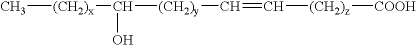
with 1≦x+y+z≦35
(5) 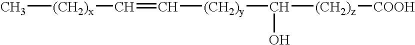
with 1≦x+y+z≦35
and HOCH₂—(CH₂)ₙ—CH═CH—(CH₂)_{y}—COOH with 2≦x+y≦36;
(6)
iv) saturated polyhydroxylated aliphatic monocarboxylic acids of formula (7):
(7) 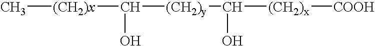
with 2≦x+y+z≦36
and the corresponding unsaturated polyhydroxylated aliphatic monocarboxylic acids;
v) saturated monohydroxylated aliphatic polyacids of formula (8):
(8) 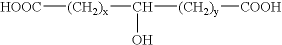
with 3≦x+y≦37
and the corresponding unsaturated monohydroxylated aliphatic polyacids; and
vi) saturated and unsaturated polyhydroxylated aliphatic polyacids.

9. The composition according to claim 8, wherein the at least one triglyceride of at least one hydroxylated carboxylic acid is chosen from triglycerides of at least one hydroxylated carboxylic acid chosen from:
12-hydroxystearic acid, α-hydroxyoctadecanoic acid, hydroxy-14-eicosenoic acid;
leucinic acid, 2-ethyl-3-hydroxycaprylic acid;
ricinoleic acid;
3-hydroxy-4-hexanoic acid, oxynervonic acid;
16-hydroxy-6-hexadecenoic acid;
9,10-dihydroxyoctadecanoic acid, 9,12-dihydroxyoctadecanoic acid, aleuritic acid, 9,10,12-trihydroxyoctadecanoic acid, hexahydroxyoctadecanoic acid, and octahydroxyoctadecanoic acid.

10. The composition according to claim 9, wherein the at least one triglyceride of at least one hydroxylated carboxylic acid is a triglyceride of ricinoleic acid.

11. The composition according to claim 1, wherein the esterification of said at least one triglyceride of at least one hydroxylated carboxylic acid is performed with at least one aliphatic monocarboxylic acid comprising from 6 to 40 carbon atoms.

12. The composition according to claim 11, wherein the at least one aliphatic monocarboxylic acid comprises from 10 to 34 carbon atoms.

13. The composition according to claim 12, wherein the at least one aliphatic monocarboxylic acid comprises from 12 to 28 carbon atoms.

14. The composition according to claim 13, wherein the at least one aliphatic monocarboxylic acid comprises from 16 to 20 carbon atoms.

15. The composition according to claim 14, wherein the at least one aliphatic monocarboxylic acid comprises 18 carbon atoms.

16. The composition according to claim 11, wherein the at least one aliphatic monocarboxylic acid is chosen from saturated and unsaturated aliphatic fatty acids.

17. The composition according to claim 16, wherein the aliphatic fatty acid is isostearic acid.

18. The composition according to claim 1, wherein the at least one aliphatic dicarboxylic acid comprises from 3 to 10 carbon atoms.

19. The composition according to claim 18, wherein the at least one aliphatic dicarboxylic acid comprises from 3 to 6 carbon atoms.

20. The composition according to claim 18, wherein the at least one aliphatic dicarboxylic acid is chosen from aliphatic dicarboxylic acids of formula HOOC—(CH₂)ₙ—COOH wherein n=1 to 4.

21. The composition according to claim 20, wherein the at least one aliphatic dicarboxylic acid is succinic acid (n=2).

22. The composition according to claim 1, wherein the at least one polyester is of the formula (I)
T₂O-(OC-D-CO-O-T₁-O)ₓ-OC-D-CO-OT₂ (I)
wherein
T₂-O- originates from the compound T₂-OH, which is a triglyceride of at least one hydroxylated carboxylic acid comprising a single free hydroxyl functional group;
-O-T₁O- originates from the compound HO-T₁-OH, which is a triglyceride of at least one hydroxylated carboxylic acid comprising two free hydroxyl functions;
-OC-D-CO- originates from the compound HOOC-D-COOH, which is said at least one aliphatic dicarboxylic acid, and
x ranges from 1 to 50.

23. The composition according to claim 22, wherein x ranges from 1 to 10.

24. The composition according to claim 23, wherein x ranges from 2 to 6.

25. The composition according to claim 22, wherein the compound T₂-OH is a triglyceride of at least one hydroxylated carboxylic acid, and said triglyceride is esterified with two molecules of the at least one aliphatic monocarboxylic acid.

26. The composition according to claim 22, wherein the compound HO-T₁-OH is a triglyceride of at least one hydroxylated carboxylic acid, and said triglyceride is esterified with one molecule of the at least one aliphatic monocarboxylic acid.

27. The composition according to claim 22, wherein the at least one polyester is of the formula (I), wherein 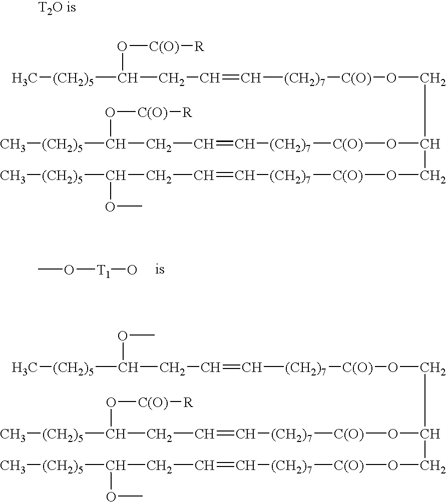
wherein R is chosen from alkyl and alkylene groups comprising from 5 to 33 carbon atoms.

28. The composition according to claim 27, wherein R is chosen from alkyl groups comprising from 7 to 17 carbon atoms.

29. The composition according to claim 27, wherein R is chosen from alkylene groups comprising from 11 to 21 carbon atoms.

30. The composition according to claim 1, wherein the at least one polyester is liquid at ambient temperature and atmospheric pressure.

31. The composition according to claim 30, wherein the at least one polyester has a viscosity of more than 500 cP (50 Pa.s) at 25° C. and/or a refractive index ≧1.47.

32. The composition according to claim 31, wherein the at least one polyester has a viscosity ranging from 900 to 10 000 cP (90 to 1 000 Pa.s) at 25° C.

33. The composition according to claim 32, wherein the at least one polyester has a viscosity ranging from 950 to 5 000 cP (95 to 500 Pa.s) at 25° C.

34. The composition according to claim 31, wherein the at least one polyester has a refractive index ranging from 1.47 to 1.55.

35. The composition according to claim 34, wherein the at least one polyester has a refractive index ranging from 1.48 to 1.55

36. The composition according to claim 1, wherein the at least one polyester is present in an amount sufficient to endow the composition with at least one property chosen from properties of non-greasiness, lubricity, gloss, stability, color retention over time, gloss retention over time, comfort, non-migration and outline definition following application of the composition.

37. The composition according to claim 1, wherein the at least one polyester is present in an amount ranging from 0.1% to 99.9% by weight of the total weight of the composition.

38. The composition according to claim 37, wherein the at least one polyester is present in an amount ranging from 1% to 99% by weight of the total weight of the composition.

39. The composition according to claim 38, wherein the at least one polyester is present in an amount ranging from 1% to 80% by weight of the total weight of the composition.

40. The composition according to claim 39, wherein the at least one polyester is present in an amount ranging from 10% to 40% by weight of the total weight of the composition.

41. The composition according to claim 40, wherein the at least one polyester is present in an amount ranging from 15% to 25% by weight of the total weight of the composition.

42. The composition according to claim 41, wherein the at least one polyester is present in an amount ranging from 20% to 25% by weight of the total weight of the composition.

43. The composition according to claim 1, wherein the at least one pasty compound has a hardness at 20° C. ranging from 0.001 to 0.5 MPa.

44. The composition according to claim 43, wherein the at least one pasty compound has a hardness at 20° C. ranging from 0.002 to 0.4 MPa.

45. The composition according to claim 1, wherein the at least one pasty compound has a liquid fraction at 23° C. ranging from 9% to 97% by weight of the total weight of the pasty compound.

46. The composition according to claim 45, wherein the at least one pasty compound has a liquid fraction at 23° C. ranging from 15% to 85% by weight of the total weight of the pasty compound.

47. The composition according to claim 46, wherein the at least one pasty compound has a liquid fraction at 23° C. ranging from 40% to 85% by weight of the total weight of the pasty compound.

48. The composition according to claim 1, wherein the at least one pasty compound has a liquid fraction at 32° C. ranging from 30% to 100% by weight of the total weight of the pasty compound.

49. The composition according to claim 48, wherein the at least one pasty compound has a liquid fraction at 32° C. ranging from 80% to 100% by weight of the total weight of the pasty compound.

50. The composition according to claim 49, wherein the at least one pasty compound has a liquid fraction at 32° C. ranging from 90% to 100% by weight of the total weight of the pasty compound.

51. The composition according to claim 1, wherein the at least one pasty compound is chosen from:
(1) polymeric and non-polymeric silicone compounds;
(2) polymeric and non-polymeric fluoro compounds;
(3) vinyl polymers;
(4) fat-soluble polyethers resulting from polyetherification of at least one diol chosen from C₂-C₁₀₀ diols; and
(5) esters.

52. The composition according to claim 51, wherein, in (3), the vinyl polymers are chosen from:
olefin homopolymers;
olefin copolymers;
hydrogenated diene homopolymers and copolymers;
linear and branched oligomers, homopolymers and copolymers of alkyl (meth)acrylates;
oligomers, homopolymers and copolymers of vinyl esters comprising at least one alkyl group chosen from C₈-C₃₀ alkyl groups; and
oligomers, homopolymers and copolymers of vinyl ethers comprising at least one alkyl group chosen from C₈-C₃₀ alkyl groups.

53. The composition according to claim 52, wherein the alkyl (meth)acrylates are chosen from those comprising at least one alkyl group chosen from C₈-C₃₀ alkyl groups.

54. The composition according to claim 51, wherein, in (4), the at least one diol is chosen from C₂-C₅₀ diols.

55. The composition according to claim 1, wherein the at least one pasty compound is chosen from hydrocarbon compounds.

56. The composition according to claim 51, wherein the at least one pasty compound is polymethyltrifluoropropylmethylalkyldimethylsiloxane.

57. The composition according to claim 51, wherein the fat-soluble polyethers are chosen from copolymers of at least one oxide chosen from ethylene oxide and propylene oxide with at least one alkylene oxide chosen from long-chain C₆-C₃₀ alkylene oxides.

58. The composition according to claim 57, wherein the ratio by weight of the at least one oxide chosen from ethylene oxide and propylene oxide to the at least one alkylene oxide in the copolymers is from 5:95 to 70:30.

59. The composition according to claim 57, wherein the fat-soluble polyethers are a block copolymer of polyoxyethylene/polydodecyl glycol.

60. The composition according to claim 51, wherein the esters are chosen from
(1) esters of an oligomeric glycerol, wherein at least one hydroxyl group of the glycerol has reacted with a mixture of fatty acids,
(2) arachidyl propionate,
(3) phytosterol esters,
(4) fatty acid triglycerides and derivatives thereof,
(5) pentaerythritol esters,
(6) non-crosslinked polyesters resulting from polycondensation of at least one acid chosen from linear and branched C₄-C₅₀dicarboxylic and polycarboxylic acids and at least one entity chosen from C₂-C₅₀ diols and polyols, and
(7) aliphatic ester esters resulting from esterification of at least one aliphatic hydroxycarboxylic ester with at least one aliphatic carboxylic acid, and mixtures thereof.

61. The composition according to claim 60, wherein, in (1), the esters of an oligomeric glycerol are chosen from diglycerol esters.

62. The composition according to claim 60, wherein, in (1), the esters of an oligomeric glycerol are chosen from condensates of adipic acid and glycerol.

63. The composition according to claim 60, wherein, in (1), the fatty acids are chosen from stearic acid, capric acid, stearic acid and isostearic acid and 12-hydroxystearic acid.

64. The composition according to claim 1, wherein the composition is in a form chosen from body makeup products, lipsticks, lipglosses, mascaras, nail varnishes, haircare and hair coloring products, and deodorants.

65. The composition according to claim 1, wherein the composition is in a form chosen from cast and compacted forms.

66. The composition according to claim 64, wherein the composition is in the form of a lipstick.

67. A method for providing a deposit on a keratin material, comprising applying to the keratin material a cosmetic composition, comprising
(i) at least one polyester resulting from esterification of at least one triglyceride of at least one hydroxylated carboxylic acid with at least one aliphatic monocarboxylic acid and with at least one aliphatic dicarboxylic acid, and
(ii) at least one pasty compound,
wherein the deposit has at least one property chosen from properties of non-greasiness, gloss, comfort, color retention over time, gloss retention over time, non-migration, effective spreading and lubricity on application, outline definition of the deposit, color intensity, and limited exudation.

68. The method according to claim 67, wherein the at least one pasty compound has a hardness at 25° C. ranging from 0.001 to 0.5 MPa.

69. The method according to claim 68, wherein the at least one pasty compound has a hardness at 25° C. ranging from 0.002 to 0.4 MPa.

70. The method according to claim 67, wherein the at least one pasty compound has a liquid fraction at 23° C. ranging from 9% to 97% by weight of the total weight of the pasty compound.

71. The method according to claim 70, wherein the at least one pasty compound has a liquid fraction at 23° C. ranging from 15% to 85% by weight of the total weight of the pasty compound.

72. The method according to claim 71, wherein the at least one pasty compound has a liquid fraction at 23° C. ranging from 40% to 85% by weight of the total weight of the pasty compound.

73. The method according to claim 67, wherein the deposit is in a form of a film.

74. A method of making a cosmetic composition, comprising including in the composition
(i) at least one polyester resulting from esterification of at least one triglyceride of at least one hydroxylated carboxylic acid with at least one aliphatic monocarboxylic acid and with at least one aliphatic dicarboxylic acid, and
(ii) at least one pasty compound.
